(19) United States
(12) Patent Application Publication
(10) Pub. No.: US 2008/0280343 A1
(43) Pub. Date: Nov. 13, 2008

(54) THROMBIN PURIFICATION

(76) Inventors: Dan Pawlak, Waunakee, WI (US); Bradley H. Knoll, Verona, WI (US); Abdel Hak Terrab, Madison, WI (US); Gerald Chesmore, Milton, WI (US); J. Foster Irwin, Alphine, UT (US)

Correspondence Address: KING PHARMACEUTICALS, INC., 400 CROSSING BOULEVARD, BRIDGEWATER, NJ, 08807, US

(21) Appl. No.: 11/927,918

(22) Filed: Oct. 30, 2007

Related U.S. Application Data
(63) Continuation of application No. 11/440,678, filed on May 24, 2006.
Application No. 11/440,678 is a continuation-in-part of application No. 11/140,374, filed on May 26, 2005.

(51) Int. Cl. C12N 9/74

(52) U.S. Cl. 435/214

(57) ABSTRACT

The invention relates to thrombin compositions with reduced levels of high molecular weight impurities. In particular, the levels of factor Va, prions and/or viral agents are greatly reduced. This invention also relates generally to methods for the preparation of thrombin having a high degree of purity and high specific activity. More specifically, the invention encompasses steps to exclude high molecular weight impurities from thrombin preparations by size exclusion filtration. In additional embodiments, the preparation of thrombin additionally includes an ion exchange filtration step. The methods of the invention are particularly suited for large scale purification of thrombin. This invention also relates generally to stabilized formulations containing thrombin compositions. More specifically, the present invention relates to stabilized, liquid formulations containing thrombin having a high degree of purity and high specific activity and methods of making and using such formulations.

This application is a continuation of U.S. application Ser. No. 11/440,678 filed May 24, 2006, which is a continuation-in-part of U.S. application Ser. No. 11/140,374 filed May 26, 2005, hereby incorporated by reference in their entirety.

### FIELD OF THE INVENTION

The invention relates generally to preparations of purified thrombin, substantially free of large molecular weight impurities, such as factor Va, prions and/or viral agents that can contribute to adverse effects in patients. This invention also relates to methods for the preparation of thrombin substantially free of viral agents, having a high degree of purity and high specific activity. More specifically, the invention encompasses methods comprising excluding high molecular weight impurities from thrombin preparations. This invention also relates generally to formulations of thrombin preparations having a high degree of purity, high specific activity and stability.

### BACKGROUND OF THE INVENTION

Thrombin is a protolytic enzyme, which appears in the blood following activation of the coagulation system as a result of proteolysis of prothrombin. Thrombin facilitates the clotting of blood by catalyzing the conversion of fibrinogen to fibrin, which forms blood clots, and releases fibrinopeptides A and B. Following a disturbance to the vascular system, the production of thrombin is central to the coagulation process.

Thrombin preparations have been approved by the FDA to be applied topically as an aid to homeostasis whenever oozing blood or minor bleeding from capillaries and small venules is accessible. Topical application of commercially available thrombin significantly speeds coagulation of the blood and significantly reduces clot times.

Studies using low purity thrombin formulations indicate that coagulopathies may occur in patients in response to exposure to low purity, topical thrombin formulations. Impurities typically present in commercially available thrombin preparations include factor Va, bovine serum albumin (BSA), and other high molecular weight proteins. Factor Va contamination of commercial bovine thrombin formulations can stimulate the production of patient anti-bovine factor Va antibodies, which can cross-react with the patient's own factor Va, thereby leading to impaired hemostasis.

The blood clotting strength of thrombin is measured in units/mL. The more concentrated the sample is, the greater the potency, the faster it will coagulate blood (or create fibrinogen). Specific activity is a ratio of the potency of a sample divided by its protein content and is expressed in units per milligram of protein.

Thrombin specific activity is dependent upon the purity of the thrombin. Highly purified thrombin shows an increase in specific activity when compared with a less pure preparation.

Previously, purification of thrombin has been generally limited to the use of conventional ion exchange chromatography. U.S. Pat. No. 5,397,704 describes a bovine preparation of thrombin that is prepared using a series of anion and cation exchange chromatography.

U.S. Pat. No. 5,151,355 describes a bovine thrombin preparation that is made by reacting one unit of prothrombin with less than 5 units of thromboplastin in the presence of calcium. The thrombin is then applied sequentially to an anion exchange agarose column and a cation exchange agarose column.

U.S. Pat. No. 4,965,203 discloses a method of purifying bovine thrombin in which the thrombin is passed through a series of ion exchange chromatography columns and then formulated with a polyol and buffers. Although the above thrombin preparations are alleged to have high specific activity, such purification schemes do not provide any means for effectively eliminating high molecular weight impurities.

United States Patent Application Publication No. 2001/0033837 discloses a method of purifying a thrombin preparation using hydrophobic interaction chromatography, optionally followed by cation exchange chromatography. Although the method of purifying thrombin includes hydrophobic interaction chromatography, the method described for purification and virus removal is not capable of achieving the virus removal and specific activity or purity encompassed by the present invention.

Accordingly, there is a need for methods that can be used to produce thrombin having a higher degree of purity. Such, the purified thrombin will have lower levels of high molecular weight impurities, such as factor Va, and a high clearance margin of viral agents and prions.

Though thrombin having a high degree of purity may be safer to use since impurities such as factor Va and BSA are reduced or eliminated, highly purified thrombin is difficult to formulate into stabilized formulations. The more pure thrombin is, the less stable it is and the more difficult it is to formulate into stabilized formulations. Therefore, there remains a need for stabilized formulations containing thrombin having a high degree of purity and high specific activity.

### BRIEF SUMMARY OF THE INVENTION

The present invention is directed to thrombin compositions and methods of preparing those compositions. As used herein, the terms formulation, composition, and preparation can be used interchangeably. The formulations, compositions, and preparations contemplated by the invention contain thrombin, preferably thrombin having enhanced purity, and may also contain additional excipients, in particular those that impart stability to the formulation, composition or preparation.

In one embodiment the present invention comprises a method for preparing thrombin having a high specific activity, enhanced purity and is substantially free of impurities, including viral particles, factor Va and prions. In accordance with the present invention, the methods for the preparations of thrombin having enhanced purity may include one or more of the following steps: size exclusion filtration, ion exchange or size exclusion chromatography, heat treatment, pH adjustment, and electromagnetic radiation. In the present invention, the source of thrombin can be bovine or human.

In yet another embodiment, the method of the present invention is capable of reducing impurities in the preparation by at least 50%, as compared to commercially available preparations such as, Thrombin-JMI®. More preferably, the method of the present invention is capable of reducing impurities in the thrombin preparation by at least 80% as compared to pre-purified or low purity bovine thrombin as described herein. In accordance with another embodiment of the present invention the method is capable of increasing the specific activity of a thrombin preparation by at least 1000%, 1200% or 1500% as compared to pre-purified or low purity bovine thrombin as described herein.

In accordance with the present invention, the size exclusion filter step is used to exclude impurities that have a molecular weight greater than 40 kDa. Preferably, the size exclusion filter is used to exclude impurities that have molecular weights ranging from 40 kDa to 300 kDa. More preferably, the size exclusion filter has a molecular weight cut-off ranging from 50 kDa to 150 kDa. In another embodiment of the invention, the size exclusion filter has a molecular weight cut-off of 50 kDa. In another embodiment the size exclusion filter has a molecular weight cut-off of 100 kDa.

The method of the present invention may also include applying a thrombin preparation to further chromatography steps, such as an ion exchange chromatography and/or size exclusion chromatography.

In another embodiment, the method of the present invention comprises applying a heat treatment to a thrombin preparation. Preferably the heat treatment includes holding the thrombin at 60° C. for 10 hours.

In another embodiment, the method of the present invention comprises lowering the pH of a thrombin preparation to about 5 or lower.

In yet another embodiment, the method of the present invention comprises the application of electromagnetic radiation to a thrombin preparation. The electromagnetic radiation can be gamma radiation or UV radiation.

The present invention encompasses a method for large-scale preparation of thrombin having enhanced purity comprising applying at least 15 L of a thrombin preparation to a size exclusion filter. In a preferred embodiment, the present invention is directed to a method for large-scale preparation of thrombin having enhanced purity comprising applying at least 15 L of a thrombin preparation to a size exclusion filter wherein the 15 L of thrombin preparation comprises about 300,000,000 units of thrombin.

The present invention is also directed to a thrombin composition. In one embodiment the thrombin composition is substantially free of impurities. In another embodiment the thrombin composition is substantially free of impurities having a molecular weight greater than 40 kDa. Preferably, the thrombin composition is substantially free of impurities having a molecular weight between 40 kDa and 300 kDa.

In yet another embodiment the thrombin composition of the present invention is substantially pure. Preferably, the thrombin composition substantially free of factor Va. More preferably, the factor Va is present at less than 0.4 μg/1000 units of thrombin. Additionally, the amount of factor Va can be measured by factor Va activity assay, ELISA, or Western Blot.

In another embodiment of the present invention, the thrombin composition has specific activity greater than 1800 u/mg of protein and is substantially free of impurities having a molecular weight greater than 40 kDa. The thrombin composition of the present invention can have a specific activity between about 1800 and 3000 u/mg of protein. Preferably, the thrombin composition can have a specific activity between about 2400 and 2500 u/mg of protein or between about 2500 and 2600 u/mg of protein, between about 2600 and 2700 u/mg of protein, between about 2700 and 2800 u/mg of protein, between about 2800 and 2900 u/mg of protein or between about 2900 and 3000 u/mg of protein. Additionally, the thrombin composition can have a specific activity greater than 3000 u/mg of protein.

The present invention is also directed to a thrombin composition substantially free of viral agents. The thrombin composition of the present invention can be substantially free of viral agents, wherein the log reduction value is greater than 3.5 per virus.

The present invention is also directed to stabilized formulations comprising thrombin having a high degree of purity and high specific activity and methods of making and using such formulations. Even though the stability of formulations can decrease as the purity of thrombin increases, the inventors have discovered stabilized formulations comprising thrombin having a high degree of purity and high specific activity.

The stabilized thrombin formulations of the present invention comprise thrombin and at least one pharmaceutically acceptable excipient.

The stabilized formulations of the present invention can contain thrombin isolated from any source including, but not limited to, bovine and human sources. Additionally, the thrombin can be any thrombin preparation or composition such as the purified thrombin compositions of the present invention or the currently commercially available Thrombin JMI®. In a preferred embodiment, the thrombin has a high degree of purity and high specific activity.

In addition to thrombin, the stabilized thrombin formulations of the present invention include an excipient. In certain embodiments, the excipients suitable for the formulations of the present invention include, but are not limited to, glycerol; polyethylene glycol; salts; aqueous solutions, such as water, acids and bases or a combination thereof.

Preferred salts include, but are not limited to, sodium chloride, sodium acetate, sodium citrate or a combination thereof.

Suitable acids and bases include, but are not limited to, hydrochloric acid or sodium hydroxide. Preferably, the formulations of the present invention have a pH of 5-9 or more preferably a pH of 6-8.

Preferably, the stabilized thrombin formulations of the present invention comprise 20-40% of glycerol by volume, 1-20% of polyethylene glycol by volume, 0.15-0.3 M concentration of sodium chloride and 0.025-0.05 M concentration of sodium acetate.

In a preferred embodiment of the present invention, the stabilized thrombin formulations of the present invention comprise purified thrombin; glycerol; polyethylene glycol; sodium chloride; sodium acetate and a pH of 6-8.

In certain embodiments the stabilized thrombin formulations of the present invention are liquid. Alternatively, the stabilized thrombin formulations of the present invention can be solid, wherein, prior to administration, the stabilized, solid thrombin formulation is dissolved or suspended in a liquid.

Additionally, the present invention is also directed to methods of administering the stabilized thrombin formulations of the present invention. Preferably, the stabilized thrombin formulations of the present invention are administered topically or to the surface of a body lumen.

The present invention is also directed to kits comprising stabilized thrombin formulations of the present invention. In certain embodiments of the invention, kits comprise stabilized thrombin formulation; a vial capable of containing the thrombin formulation; and a needle.

In other embodiments of the invention, kits comprise a thrombin formulation and a device that is capable of spraying the thrombin formulation. Suitable spray devices include, but are not limited to, a spray tip or a spray pump.

The present invention is directed to stabilized thrombin formulations which maintain at least 60% of their initial potency for a period of two years. In preferred embodiments the formulations maintain at least 65%, 70%, 75%, 80%, 85%, 90%, 95% of their initial potency. The invention is particularly directed to formulations that maintain at least 80%, 85%, 90%, or 95% of their initial potency after 3 months, at least 70%, 80%, 85%, or 90%, 95% of their initial potency after 6 months, at least 70%, 80%, 85%, 90%, or 95% of their initial potency after 9 months, at least 70%, 80%, 85%, 90%, or 95% % of their initial potency after 12 months, and at least 60%, 70%, 80%, 85%, 90%, or 95% % of their initial potency after 18 months. The present invention is also directed to stabilized thrombin formulations that maintain at least 70%, 75%, 80%, 85%, 90%, or 95% of initial label claim potency for a period of two years. The invention is directed to formulations that maintain at least 70%, 75%, 80%, 85%, 90%, or 95% of their initial label potency after 3 months, at least 70%, 75%, 80%, 85%, 90%, or 95% of their initial label potency after 6 months, at least 70%, 75%, 80%, 85%, 90%, or 95% of their initial label potency after 9 months, at least 70%, 75%, 80%, 85%, 90%, or 95% of their initial label potency after 12 months, at least 70%, 75%, 80%, 85%, 90%, or 95% of their initial label potency after 18 months.

### BRIEF DESCRIPTION OF THE DRAWINGS

FIG. 1 shows a flow diagram of all of the steps used to prepare a thrombin preparation, in accordance with the method of the present invention.

FIG. 2 shows a Sodium Dodecyl Sulfate Polyacrylamide Gel Electrophoresis (SDS-PAGE) comparison of Thrombin-JMI® after the addition of the purification process of the present invention (lanes 7, 8 and 9) to Thrombin-JMI® as currently manufactured (lanes 4 and 5) and the retentive of the size exclusion filtration, showing the high molecular weight impurities (lane 11).

FIG. 3 shows a method of making the stabilized thrombin formulations of the present invention.

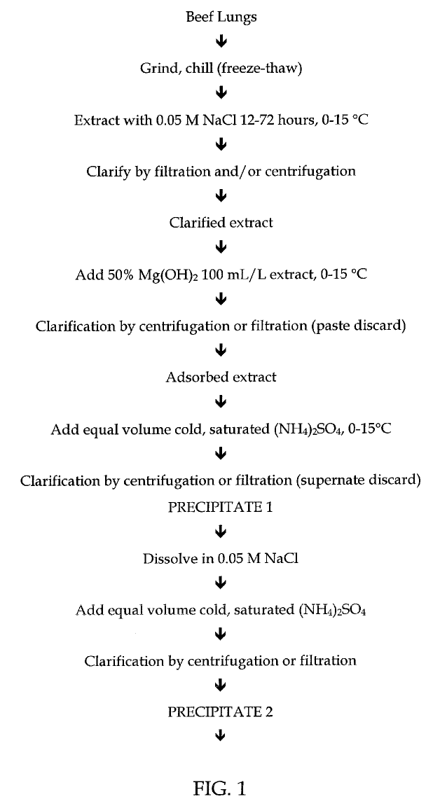

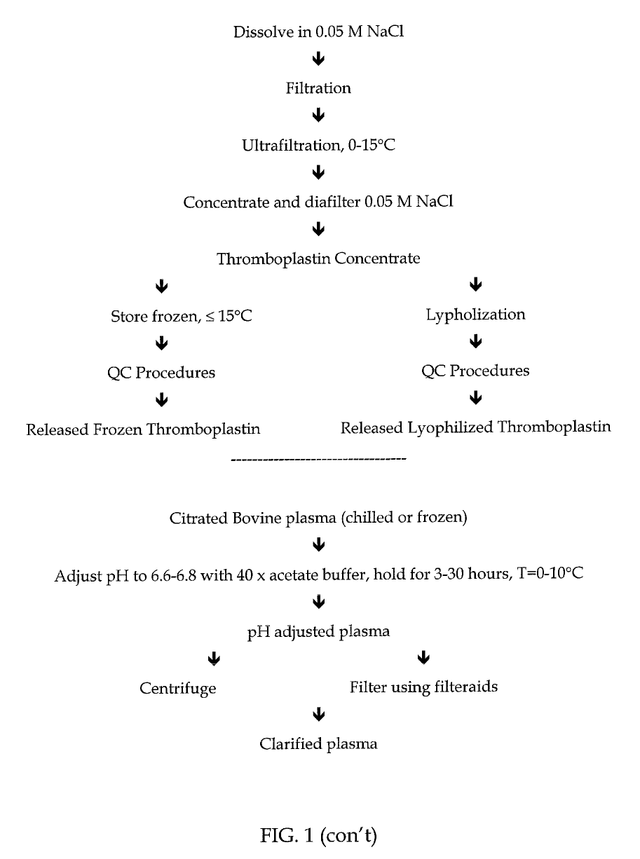

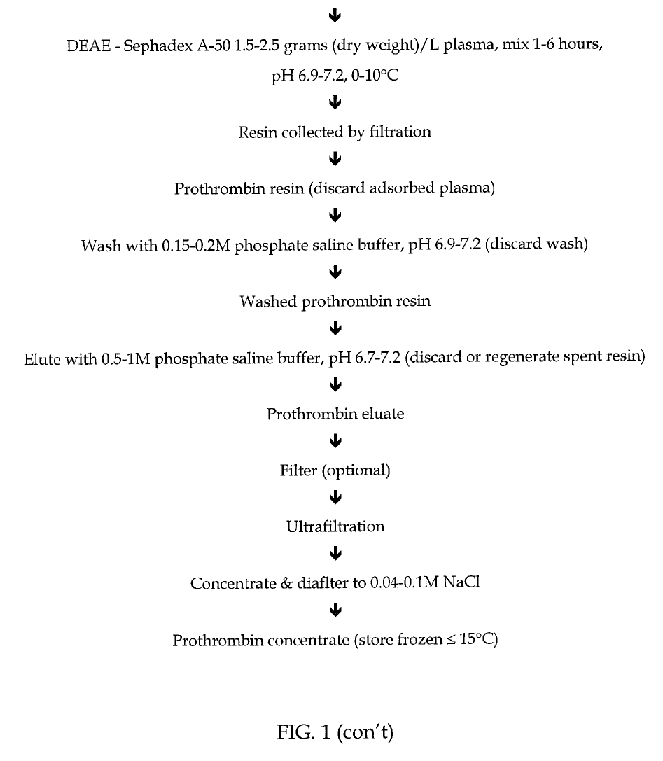

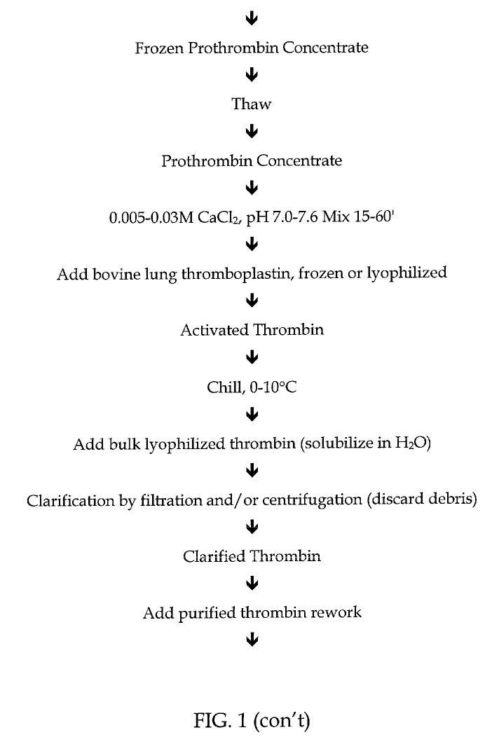

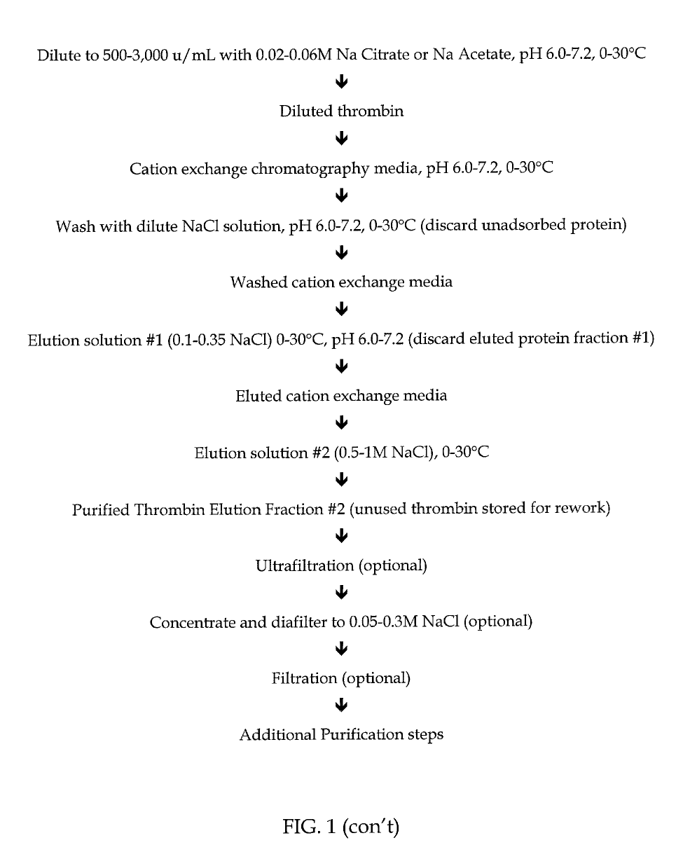

### DETAILED DESCRIPTION OF THE INVENTION

The present invention is based on the discovery that the use of size exclusion filtration alone or in combination with other purification steps for the purification of thrombin provides substantial benefits over prior art thrombin purification methods. The methods of purifying thrombin of the present invention provide thrombin that is significantly more pure and safe, due to the substantial removal or elimination of high molecular weight impurities. The methods of the present invention also provide a high degree of viral clearance, along with consistency, reliability, and ease of use.

The present invention encompasses applying purifying steps to a thrombin preparation and recovering the purified thrombin. These steps include, but are not limited to, chromatographic purification; applying the thrombin preparation to a size exclusion filter; applying the thrombin preparation to an ion exchange filter; lowering the pH; or irradiating the thrombin preparation with electromagnetic radiation. Though the invention is based on the discovery of the use of size exclusion filtration, such purification steps may be applied independently or in combination.

Furthermore, the methods are amenable to large scale, commercial production and purification. The methods of the present invention can yield large quantities of thrombin substantially free of impurities, having a molecular weight of greater than 40 kDa, and viral agents.

The present invention also contemplates the addition of one or more excipients to thrombin, resulting in a thrombin formulation that is more stable than currently available thrombin formulations. As such, the present invention solves many of the problems of the thrombin formulations in the prior art. Without being bound by any particular theory, the ability to stabilize the highly purified thrombin compositions of the present invention can result in consistent and effective treatment. Preferably, the stabilized thrombin formulations of the present invention comprise thrombin having a high degree of purity and specific activity and at least one pharmaceutically acceptable excipient. The present invention also encompasses liquid, stabilized thrombin formulations.

Thrombin from any source can be used in the compositions, formulations and methods of the present invention. Examples of sources of thrombin suitable for use in the compositions, preparations and methods of the present invention include, but are not limited to thrombin isolated from bovine or human sources. Also, commercial sources of thrombin, such as Thrombin JMI® can be used in the present invention.

Also thrombin of any purity level or thrombin resulting from any preparation can be used. For example, pre-purified thrombin as described herein can be used in the compositions, formulations and methods of the present invention. Additionally, thrombin resulting from natural or recombinant preparations is suitable for the present invention.

The present invention includes methods for the preparation of thrombin with increased purity, increased specific activity, and increased safety due to low impurity levels of impurities, such as factor Va, prions, and viral particles.

In certain embodiments, the methods of the present invention increase the purity of a thrombin preparation by more than 30%, more than 50%, more than 75%, or more than 90%, as compared to Thrombin-JMI®, or other purified thrombin.

Another way purity may be quantified is by measuring the specific activity of thrombin. Specific activity of thrombin can be measured by standard assays known in the art, including clotting assays and chromogenic assays (See, e.g., Gaffney et al., 1995, Thromb Haemost 74: 900-903).

In one embodiment, the methods of the invention provide for thrombin preparations where the specific activity of the thrombin preparation is increased by at least 1000%, at least 1200%, at least 1500%, or at least 1800%, as compared to pre-purified thrombin. The thrombin compositions of the present invention have a high specific activity; preferably the specific activity of the thrombin compositions of the present invention is greater than 1800 u/mg of protein.

The present invention methods of the invention provide for thrombin preparations having a specific activity ranging from about 1800 u/mg and 3000 u/mg, more preferably between about 1800 u/mg and 2400 u/mg. In other embodiments, the specific activity is between about 2400 u/mg and 2500 u/mg, between about 2500 u/mg and 2600 u/mg, or between about 2600 u/mg and 2700 u/mg, between about 2700 and 2800 u/mg of protein, between about 2800 and 2900 u/mg of protein or between about 2900 and 3000 u/mg of protein. In certain embodiments, the thrombin has a specific activity greater than 3000 u/mg. Preferably, after size exclusion filtration the specific activity rises from ≧about 1500 u/mg to ≧about 2300 u/mg.

Using certain methods of the invention, viral agents and/or high molecular weight impurities are reduced by at least 50%, at least 60%, at least 75%, at least 80%, at least 85%, at least 90%, at least 95%, or at least 99%. In a preferred embodiment, high molecular weight impurities and/or viral particle impurities in the thrombin preparation are reduced by at least 80%.

The present invention also provides methods for purifying thrombin comprising excluding molecules having a higher molecular weight than thrombin. Methods for purifying thrombin to eliminate at least 50%, at least 55%, at least 60%, at least 65%, at least 70%, at least 75%, at least 80%, at least 85%, at least 90%, at least 95% and at least 99% of impurities having a higher molecular weight than thrombin are provided. In achieving elimination of higher molecular weight impurities, it is desirable to achieve recoveries of thrombin of at least 70%, at least 75%, at least 80%, at least 85%, at least 90%, at least 95%, or at least 99%.

Also in certain embodiments of the methods of the present invention, the recovery of thrombin is also greater than 80%, greater than 85%, greater than 90%, or greater than 95%.

The methods of the present invention include applying purification steps to a thrombin preparation and recovering the purified thrombin. The purification steps of the present invention include applying a thrombin preparation to a size exclusion filter; chromatographic purification; applying the thrombin preparation to an ion exchange filter; lowering the pH; or irradiating the thrombin preparation with electromagnetic radiation. Such steps may be applied independently or in combination.

According to the methods of the present invention, recovery and purification of thrombin preparations can be achieved by excluding impurities using methods involving separation techniques based on molecular weight. In general, any method involving separation based on molecular weight can be used, including size exclusion filtration and chromatography. In certain embodiments where the methods of the present invention use size exclusion filtration, it is preferable that filter pores are large enough to allow the passage of the thrombin molecules, but small enough to retain many impurities, including large protein impurities and viruses.

Since thrombin has a molecular weight of approximately 40 kDa, it is preferable that, in certain embodiments, the methods of the invention comprise applying a thrombin preparation to a size exclusion filter capable of excluding impurities that have a molecular weight greater than 40 kDa in size from said thrombin preparation. In a preferred embodiment, the size exclusion filter is capable of excluding impurities that have a molecular weight ranging from 40 kDa to 300 kDa.

Thus, size exclusion filters of molecular weight cut-offs, i.e., exclusion limit, of 50, 100, 150, 300 kDa or greater can be used. In one embodiment, the size exclusion filter has a molecular weight cut-off ranging from 40 kDa to 300 kDa. In another embodiment, the size exclusion filter has a molecular weight cut-off ranging from 50 kDa to 300 kDa. In yet another embodiment, the size exclusion filter has a molecular weight cut-off ranging from 50 kDa to 150 kDa. In a preferred embodiment, the size exclusion filter has a molecular weight cut-off of 50 kDa. In a more preferred embodiment, the size exclusion filter has a molecular weight cut-off of 100 kDa. This step can also optionally include the application of dia-filtration to maximize thrombin recovery.

In preferred embodiments, the size exclusion filters will have pore sizes with a molecular weight cut-off of around 100 kDa. Preferably, a size exclusion filter suitable for the present invention also effectively reduces bacterial agents and endotoxins.

In certain embodiments, size exclusion filters are made of modified polyethersulfone on a highly porous polyolefin backing. Also, the filter used can be a tangential flow filter. One example of a filter that can be used in this invention is the Omega™ 100K VR manufactured by PALL FILTRON Corporation.

Other size exclusion filters that may be used in accordance with this invention include, but are not limited to the Viresolve/70 manufactured by Millipore Corporation; VirA/Gard 500 manufactured by A/G Technology, Corporation, and Ultipor DV20 manufactured by Pall Corporation.

With size exclusion filtration the large molecules, including viral impurities, get retained by the pores in the membrane. The membrane can be discarded after use or, in the alternative, the membrane can be reused. Membranes that result in a high enough log reduction are considered acceptable, and can be used. Each log reduction is a reduction of 90%. Other tests can also be performed on the filter to ensure the filter has an acceptable pore size range. In certain embodiments of the methods of the present invention the viral clearance is at a log reduction value (LRV) greater than 3.5, preferably greater than 4.0, more preferably greater than 4.5. In certain embodiments, prion clearance is at a log reduction value (LRV) greater than 3.5, preferably greater than 4.0, more preferably greater than 4.5.

In certain embodiments of the invention, initial volumes of 50 mL, 100 mL, 150 mL, 200 mL, 250 mL, 300 mL, 350 mL, 400 mL, 450 mL, 500 mL, multiples thereof, or more are applied to a size exclusion filter. The invention also encompasses methods comprising applying at least 300 mL of a thrombin preparation to a size exclusion filter.

The invention also encompasses methods for large scale, commercial purification of thrombin comprising applying at least 40 L of a thrombin preparation to a size exclusion filter, preferably at least 60 L of a thrombin preparation to a size exclusion filter, most preferably at least 90 L of a thrombin preparation to a size exclusion filter. In certain embodiments, the volume of thrombin preparation applied to the size exclusion filter depends on the surface area of the filters used and/or the number of filters used. In preferred embodiments of the invention, initial volumes of 40 L to 60 L, 60 L to 80 L, 80 L to 100 L, over 100 L, or more and multiples thereof are applied to a size exclusion filter.

The certain methods of the present invention are particularly suited to large-scale purifications of thrombin. As such, in preferred embodiments of invention, initial volumes of 15 L to 20 L and multiples thereof are applied to a size exclusion filter. In one embodiment when 15 L of thrombin preparation is applied to the size exclusion filter, the thrombin preparation comprises 300,000,000 units of thrombin.

Used alone or in conjunction with size exclusion filtration, the use of ion exchange filtration also provides substantial benefits to thrombin purification. Ion exchange filtration provides a high degree of viral clearance along with consistency, reliability and ease of use.

In certain embodiments, methods of the present invention may further comprise applying the thrombin to an ion exchange filter. Ion exchange filtration is a separation method which filters solutes based on their electronic charge. Ion exchange filters contain charge centers on the ion exchange membrane. When the sample is passed through the filter, the charged compounds in the sample will adsorb onto the charge centers on the membrane. A filter is selected that has a positive charge and that will filter out charged protein and viral impurities from the thrombin preparation. Viruses with the same net charge as the filter will not bind to the resin and will be cleared in the breakthrough.

Ion exchange filters used in accordance with this invention are preferably charged positively, whereas ion exchange chromatography resins typically used for thrombin purification are charged negatively. Ion exchange filters are efficient for removing nucleic acids. In a preferred embodiment, an ion exchange filer has pendant quaternary amine groups. A preferred ion exchange filter that can be used with this invention is the Mustang™ Q filter manufactured by the Pall Corporation. Another ion exchange filter that can be used is the Cuno Zeta Plus VR05.

Application of moderate heat is also a suitable step that can be used in the methods of the present invention for the purification of thrombin. Application of moderated heat can also be used alone or in connection with size exclusion filtration or chromatography as well as any of the other purification steps discussed herein. Any type of heat can be applied to thrombin, from any source, for any length of time so long as the viral impurities are inactivated and the thrombin still maintains a high specific activity.

In certain embodiments the thrombin can be heated to 40° C. to 100° C. In preferred embodiments the thrombin is heated to about 60° C. The heat treatment can be applied to the thrombin for any length or time. For example, thrombin can be heated for 1 to 60 minutes or the thrombin can be heated for 1, 2, 3, 4, 5, 6, 7, 8, 9 or 10 hours. In a preferred embodiment, a heat treatment is applied to thrombin for 10 hours at 60° C. is used.

Another purification step that can be used to in the methods of the present invention is adjusting the pH of the thrombin. The pH of the thrombin can be adjusted to any level so long as the resulting thrombin composition has low amounts of viral impurities. For example, lowering the pH of the thrombin is effective in inactivating viral impurities. In certain embodiments, the pH of the thrombin is adjusted to below about 5 or below about 4. However, lowering the pH of the thrombin can result in some loss of thrombin activity.

Yet another purification step that can be used to in the methods of the present invention is the application of gamma or electromagnetic radiation, or UV light. Application of radiation to thrombin can also be used alone or in combination with any of the other purification steps described herein.

The inventors of the present invention have found that electromagnetic and gamma radiation are powerful and robust virus inactivation tools. Reportedly, gamma irradiation is effective against a wide variety of viruses. However, less than 70% recovery can be obtained in the case of commercially available thrombin.

Application of UV light can also be used alone of in combination with the other purification steps of the present invention and is also an effective virus inactivation procedure. However, some loss of thrombin activity is observed with this as well method. The inventors noted that short exposure periods may result in less of a loss of activity.

The present invention is also directed to thrombin compositions purified by the above methods. The thrombin composition of the present invention have increased purity, high specific activity and low impurity levels, including low levels of impurities, such as factor Va, prions, and viral particles.

The thrombin compositions of the present invention have a high specific activity; preferably the specific activity of the thrombin compositions of the present invention is greater than 1800 u/mg of protein. The present invention encompasses thrombin compositions having a specific activity ranging from about 1800 u/mg and 3000 u/mg, more preferably between about 1800 u/mg and 2400 u/mg. In other embodiments, the specific activity is between about 2400 u/mg and 2500 u/mg, between about 2500 u/mg and 2600 u/mg, or between about 2600 u/mg and 2700 u/mg, between about 2700 and 2800 u/mg of protein, between about 2800 and 2900 u/mg of protein or between about 2900 and 3000 u/mg of protein. In certain embodiments, the thrombin has a specific activity greater than 3000 u/mg.

Also the thrombin compositions of the present invention are substantially free of high molecular weight impurities, including, factor Va, bacterial agents, prions and viral agents. As used herein, a compositions that are “substantially free” of a high molecular weight impurities means that the compositions contain less than about 5-20% by weight, preferably less than about 15% by weight, more preferably less than about 10% by weight. As used herein, compositions that are “substantially pure” contain less than 5% of the high molecular weight impurities by weight, and most preferably less than about 3% by weight of the high molecular weight impurities.

In a preferred embodiment, the thrombin compositions of the present invention are substantially free of impurities having a molecular weight of greater than 40 kDa. In another preferred embodiment, the thrombin is substantially free of impurities having a molecular weight in the range of 40 kDa to 300 kDa. Examples of high molecular weight impurities include factor Va (heavy chain (mol. wt.=105 kDa) and light chain (mol. wt.=71 kDa/74 kDa)) and bovine serum albumin (BSA; mol. wt.=66 kDa).

In another specific embodiment, the invention provides thrombin compositions substantially free of viral particle impurities. Viral particle impurities are also examples of high molecular weight impurities. Viruses that can be removed by the methods of the present invention include, but are not limited to, bovine viral diarrhea virus (BVDV), pseudorabies virus (PRV), encephalomyocarditis virus (EMCV), bovine parvovirus (BPV), canine parvovirus (CPV), stickleback virus (SBV), tick-borne encephalitis virus (TBEV), equine rhinovirus 1 (ERV-1), human immunodeficiency virus 1 (HIV-1), hepatitis A (HAV), hepatitis B (HBV), and hepatitis C (HCV). Viruses can be detected by a variety of antibody based assays, including ELISAs and nucleic acid based assays, including PCR and hybridization assays.

In a specific embodiment, the invention provides thrombin compositions substantially free of Factor Va. In some embodiments, factor Va is reduced by at least 50%, at least 55%, at least 60%, at least 65%, at least 70%, at least 75%, at least 80%, at least 85%, at least 90%, at least 95%, or at least 99%. In certain embodiments of the invention, after size exclusion filtration, factor Va can barely be detected in the concentrated sample (before it is diluted into a final formulation) and is typically not detected at all in the final formulation. In other embodiments, the amount of factor Va is reduced to less than 0.4, less than 0.35, less than 0.3, less than 0.25, less than 0.2, less than 0.15, less than 0.1, less than 0.02 μg/1000 units of thrombin or any other currently undetectable amount.

Preferably, the absence or reduced levels of factor Va is determined by routine methods known in the art, e.g., chromatographic methods, including gel electrophoresis, factor Va activity assays and antibody based assays.

In a preferred embodiment, the thrombin compositions of the present invention have a specific activity greater than 1800 u/mg of protein and are substantially free of high molecular weight impurities.

Thrombin purified by the methods of the present invention can be further formulated for clinical use. Thrombin formulations of the present invention preferably are more stable than currently available thrombin formulations. Additionally, in certain embodiments the stable thrombin formulations of the present invention are liquid.

In certain embodiments, the stabilized formulations of the present invention comprise: thrombin, wherein the thrombin is substantially free of impurities; and at least one pharmaceutically acceptable excipient. In one such embodiment, the thrombin is bovine thrombin.

In certain embodiments, the stabilized formulations of the present invention comprise thrombin, at least one polymer, at least one alcohol, at least one salt and an appropriate amount of an acid and/or base to adjust the pH to the desired range.

In one embodiment the stabilized formulations of the present invention comprise thrombin, glycerol, polyethylene glycol, sodium acetate and sodium chloride and either hydrochloric acid or sodium hydroxide or both to adjust the pH to between 5-8.

In a preferred embodiment the stabilized formulations of the present invention comprise thrombin, about 30% glycerol by volume, about 10% polyethylene glycol by volume, 0.025-0.05 M concentration of sodium acetate and 0.15-0.3 M concentration of sodium chloride and either hydrochloric acid or sodium hydroxide or both to adjust the pH to a range of 6-7.

The purified thrombin of the present invention may be stored at 0-10° C. for up to 48 hours prior to formulation and/or sterile processing. In a preferred embodiment sterilization of the formulation of the invention is achieved using a 0.2 micron sterile filter. The formulation of the present invention may be stored at 25° C. for up to 2 years without sterile processing. The present invention is directed to stabilized thrombin formulations which maintain at least 60% of their initial potency for a period of two years. In preferred embodiments the formulations maintain at least 65%, 70%, 75%, 80%, 85%, 90%, or 95% of their initial potency. The invention is particularly directed to formulations that maintain at least 80%, 85%, 90%, or 95% of their initial potency after 3 months, at least 70%, 80%, 85%, 90%, or 95% of their initial potency after 6 months, at least 70%, 80%, 85%, 90%, or 95% of their initial potency after 9 months, at least 70%, 80%, 85%, 90%, or 95% % of their initial potency after 12 months, and at least 60%, 70%, 80%, 85%, 90%, or 95% % of their initial potency after 18 months. The present invention is also directed to stabilized thrombin formulations that maintain at least 70%, 75%, 80%, 85%, 90%, 95% of initial label claim potency for a period of two years. The invention is directed to formulations that maintain at least 70%, 75%, 80%, 85%, 90%, or 95% of their initial label potency after 3 months, at least 70%, 75%, 80%, 85%, 90%, or 95% of their initial label potency after 6 months, at least 70%, 75%, 80%, 85%, 90%, or 95% of their initial label potency after 9 months, at least 70%, 75%, 80%, 85%, 90%, or 95% of their initial label potency after 12 months, at least 70%, 75%, 80%, 85%, 90%, or 95% of their initial label potency after 18 months.

Suitable pharmaceutically acceptable excipients include any excipient that aids in the stabilization of the thrombin formulations of the present invention. The pharmaceutically acceptable excipients that are suitable for the present invention can function as diluents, buffers, stabilizers, surfactants, chelating agents, preservatives.

Suitable pharmaceutically acceptable excipients include, but are not limited to, aqueous liquids, such as water, acids and bases; organic solvents, such as alcohols; polymers; salts or a combination thereof.

Suitable acids include, but are not limited to, hydrochloric acid, hydrobromic acid, sulfuric acid, nitric acid, formic acid, acetic acid, citric acid, and phosphoric acid. The preferably acid is hydrochloric acid.

Suitable bases include, but are not limited to sodium hydroxide, potassium hydroxide and ammonium hydroxide. The preferably base is sodium hydroxide.

Acids and bases included in the stabilized, liquid thrombin formulations of the present invention can be used to adjust the pH of the stabilized, liquid thrombin formulations. The pH of the stabilized, liquid thrombin formulations of the present invention can be adjusted to any pH that stabilizes the thrombin formulations of the present invention. In certain embodiments the pH of the thrombin formulation is between 4 and 9. Preferably, the pH of the thrombin formulations of the present invention is between 5 and 8. More preferably, the pH of the thrombin formulations of the present invention is between 5.5 and 7.7. In one preferred embodiment the pH of the thrombin formulations of the present invention is 6.7±0.1.

Suitable alcohols include, but are not limited to polyhydric alcohols, such as glycerol ethylene glycol, propylene glycol, butylene glycol, 1,6-hexylene glycol, neopentyl glycol, diethylene glycol, trimethylolpropane, and pentaerythritol; The preferred alcohol is glycerol.

The amount of alcohol in the stabilized, liquid thrombin formulations of the present invention can be 0 to 80% by volume. In certain embodiments the amount alcohol, for example glycerol is 10-50% by volume. In preferred embodiments the amount of alcohol, for example glycerol, is 20-40% or 25-35% or 30% by volume.

Suitable polymers include, but are not limited to, polyethylene glycol, styrene-isobutylene-styrene, polyurethanes, silicones, polyesters, polyolefins, polyisobutylene, ethylene-alphaolefin copolymers, acrylic polymers and copolymers, vinyl halide polymers, polyvinyl ethers, polyvinylidene halides, polyacrylonitrile, polyvinyl ketones, polyvinyl aromatics, polyvinyl esters, copolymers of vinyl monomers, copolymers of vinyl monomers and olefins, polyamides, alkyd resins, polycarbonates, polyoxymethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM rubbers, fluorosilicones, polysaccharides, phospholipids, or a combination thereof. The preferred polymer is polyethylene glycol. A very preferred polymer is polyethylene glycol 200-400.

The amount of polymer in the stabilized, liquid thrombin formulations of the present invention can be 0 to 50% by volume. In certain embodiments the amount polymer, for example polyethylene glycol, is 1-30% by volume. In preferred embodiments the amount of polymer, for example, polyethylene glycol, is 1-20% or 5-15% or 10% by volume. Suitable salts include, but are not limited to, calcium, potassium or sodium chloride, bromide, and the like; calcium, potassium, cesium or sodium acetate; potassium, cesium or sodium citrate; potassium, cesium or sodium nitrate; and potassium, cesium or sodium formate. Preferred salts are sodium acetate and sodium chloride.

The concentration of salts in the formulations of the present invention can be 0 to 0.5 M. In certain embodiments, the concentration of salt is 0.01 to 0.45 M. In other embodiments the salt concentration is less than 0.3 M. In preferred embodiments, the salt is a combination of sodium chloride and sodium acetate, wherein the concentration of sodium chloride is 0.15-0.3 M and the concentration of sodium acetate is 0.025-0.05 M. Another preferred range of sodium chloride is 0.28 to 0.32 M.

Thrombin can be tested for potency or activity can be tested using assays known in the art. Once such method is based on clot time measurements. Clot time measurements can be determined using an ACL 7000 coagulation timer. The measured clot times are converted into u/mL using a double log regression of the standard curve's clot time vs. u/mL. The u/mL value is multiplied by the dilution factor to obtain the actual potency of the sample. Assay results may have some variability is due to a combination of factors including pipetting techniques, different ACL machines, and viscosity of the sample tested.

The present invention also encompasses kits comprising stabilized thrombin formulations of the present invention. The kits may include a vial which contains the stabilized thrombin formulations of the present inventions. While in the vials the stabilized thrombin formulation should meet the following product specifications:

| | | Shelf Life (2 years at room |
|---|---|---|
| Specification | Release | temperature) |
| Potency | NLT 1,400 u/mL | NLT 900 u/mL |
| Fill Volume | | |
| 5,000 u/vial | NLT 5.0 mL | NLT 5.0 mL |
| 10,000 u/vial | NLT 10.0 mL | NLT 10.0 mL |
| 20,000 u/vial | NLT 20.0 mL | NLT 20.0 mL |
| pH | 5.9-7.5 | 5.9-7.5 |

| | |
|---|---|
| Sterility | Meets the requirements of USP and 21 CFR |
| | 610.12 sterility test |
| General Animal Safety | Meets the requirements of modified |
| | 21 CFR 610.11 |

In certain embodiments the kits of the present invention comprise the stabilized thrombin formulations; a vial capable of containing the thrombin formulation; and a needle. The kits may also include at least one sponge.

In other embodiments the kits of the present invention are spray kits wherein the kits comprise a stabilized thrombin formulation and a device that is capable of spraying the thrombin formulation. Suitable spraying devices include a spray tip or a spray pump and an actuator. Additionally, the spray kits can further comprise a needle.

In connection with the spray kits the stabilized thrombin formulation can also be contained in a vial. The vials containing the stabilized, liquid thrombin formulation included in the spray kits should meet the following product specifications:

| | | Shelf Life (2 years at room |
|---|---|---|
| Specification | Release | temperature) |
| Potency | NLT 1,400 u/mL | NLT 900 u/mL |
| Fill Volume | | |
| 5,000 u/vial | NLT 5.0 mL | NLT 5.0 mL |
| 10,000 u/vial | NLT 10.0 mL | NLT 10.0 mL |
| 20,000 u/vial | NLT 20.0 mL | NLT 20.0 mL |
| pH | 5.9-7.5 | 5.9-7.5 |

| | |
|---|---|
| Sterility | Mets the requirements of USP and 21 CFR |
| | 610.12 sterility test after ethylene oxide |
| | sterilization. |
| General Animal Safety | Mets the requirements of modified 21 CFR |
| | 610.11 after ethylene oxide sterilization. |

The present invention also relates to methods of making stabilized thrombin formulations. In certain embodiments a method for making stabilized thrombin formulations comprises (a) enhancing the purity of thrombin; and (b) adding at least one pharmaceutically acceptable excipient to the purified thrombin.

The methods of making the stabilized formulations of the present invention can further comprise adjusting the pH with an acid or a base.

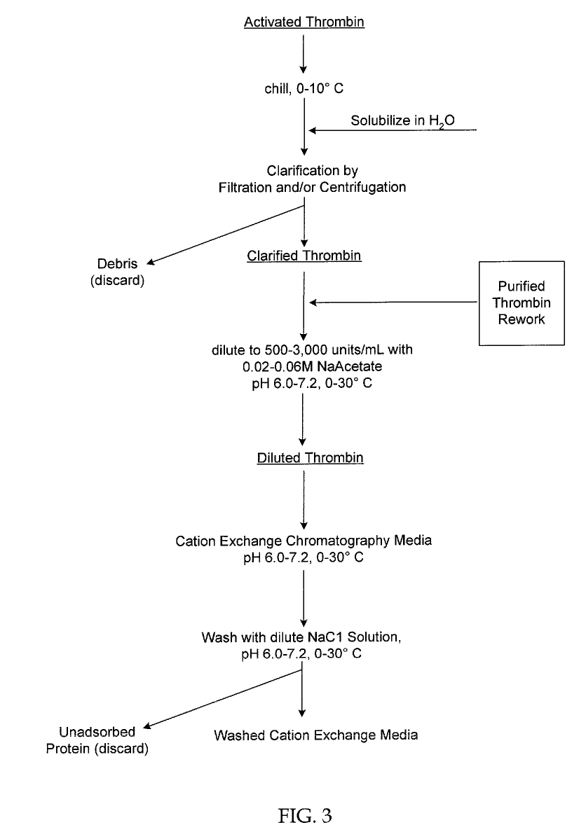

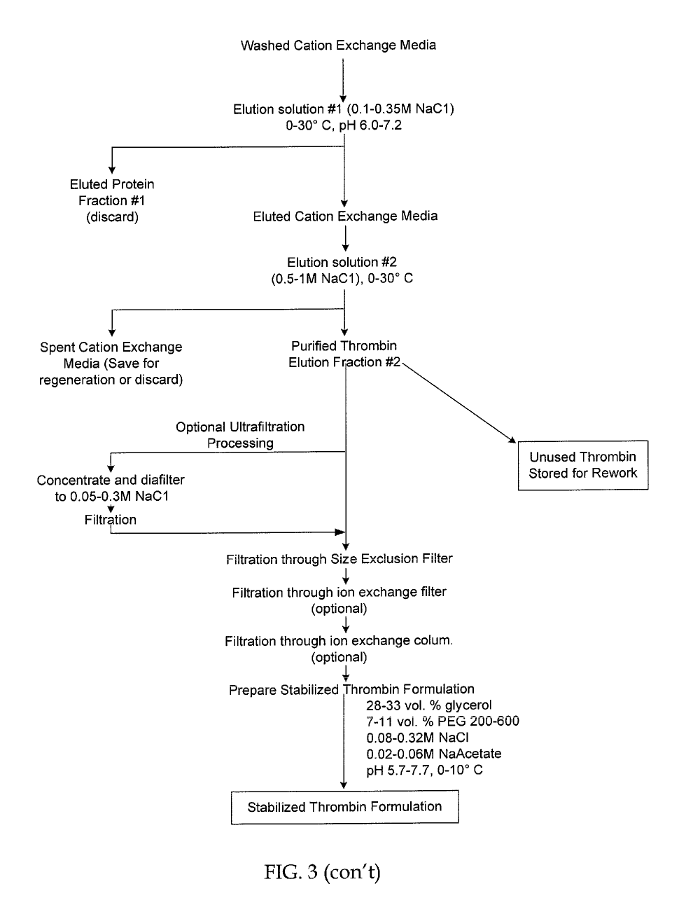

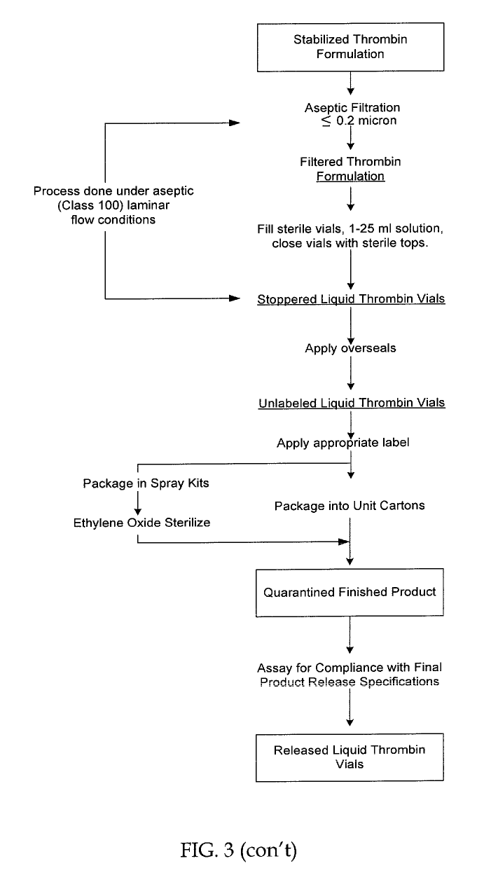

FIG. 3 shows a flow chart which describes a method of making stabilized thrombin formulations of the present invention. After preparing a thrombin composition having enhanced purity and specific activity, 28-33% by volume of glycerol, and 7-11% by volume of polyethylene glycol having a molecular weight of between 200-600 are added to the purified thrombin. Sodium chloride and sodium acetate are added until the molar concentration of sodium chloride is 0.08-0.32 M and the molar concentration of sodium acetate is 0.02-0.06 M. The pH is adjusted to 5.7 to 7.7. The stabilized thrombin formulation is then cooled to 0-10° C.

The stabilized thrombin formulation can then be sterilized and stored in labeled vials.

Additionally, the present invention is also directed to methods of administering the stabilized thrombin formulations. Preferably the stabilized thrombin formulations of the present invention are administered topically.

In certain embodiments the method comprises (a) drawing the thrombin formulation into a syringe; (b) forcing the thrombin formulation through the syringe; and (c) flooding the surface of a body lumen with the thrombin formulation.

In other embodiments, the method comprises spraying the topical thrombin formulation onto the surface of a body lumen.

In yet other embodiments the method comprises (a) saturating a sponge with the thrombin formulation; and (b) applying the sponge to the surface of a body lumen.

The description contained herein and the following examples are for purposes of illustration and not for purposes of limitation. Changes and modifications may be made to the embodiments of the description and still be within the scope of the invention. Furthermore, obvious changes, modifications or variations will occur to those skilled in the art.

### EXAMPLES

### Example 1

### Preparation of Pre-Purified Thrombin

Preparation of thromboplastin: fresh Bovine Lung is ground in conventional grinding equipment. Ground Bovine Lung may be used immediately or stored frozen in poly-lined containers at <−15° C. The ground lung is suspended in dilute sodium chloride at about 0-15° C. and extracted for about 12-72 hours. The lung suspension is filtered through coarse fabric and/or, alternatively, by centrifugation, and the liquid extract is collected.

While under agitation, approximately 100 mL of an approximately 50% suspension of magnesium hydroxide gel is added per liter of lung extract and thoroughly mixed. The suspension is centrifuged or, alternatively, filtered with filter aids and the centrifugate or filtrate collected. The adsorbed lung extract is fractionated by adding, under agitation at about 0-15° C., approximately one liter of cold saturated ammonium sulfate per liter of lung extract and mixed for about 15-480 minutes. The insoluble paste is harvested by centrifugation or, alternatively, by filtration with filter aids.

The paste is resolubilized in about 0.25 to 1 liter of cold dilute sodium chloride per liter of starting lung extract. The fractionated lung extract is reprecipitated by adding under agitation at about 0-15° C., approximately one liter of cold saturated ammonium sulfate per liter of solution, and mixed for about 15-480 minutes. The insoluble paste is harvested by centrifugation or, alternatively, by filtration with filter aids. The second paste is resolubilized in cold dilute sodium chloride and clarified by filtration.

The thromboplastin solution is concentrated in a suitable ultrafilter system to about 10-50% of the original volume and then diafiltered to remove detectable ammonium sulfate. Ultrafiltration is a process whereby a solution with a solute of molecular size that is much greater than that of the solvent is separated from the solvent by the application of hydraulic pressure. The hydraulic pressure forces the solvent through a suitable membrane and concentrates the solute.

The diafiltration is conducted by adding 8 or more volumes of 0.05 M NaCl or until the permeate passed the barium chloride test. Diafiltration is a process of separating microsolutes from a solution of larger molecules by ultrafiltration with a continuous addition of solvent. The concentrate is then further optionally concentrated and the ultrafiltration completed. The ultrafiltration system is rinsed with several liters of chilled dilute sodium chloride and this wash is added to the concentrate. The pH of the concentrate is adjusted to about 7.0 with dilute hydrochloric acid or dilute sodium hydroxide. The resulting Thromboplastin is stored at about −15° C. or colder in sealed plastic containers.

Fresh Bovine Plasma, citrated, is received either frozen or chilled in a tank truck. If received frozen, the plasma is generally stored frozen until thawed for usage. Thawed plasma is maintained at 0-10° C. in stainless steel tanks. The pH of the plasma is adjusted with buffered acetic acid to about 6.6-6.8 and held for about 3-30 hours. At the end of the hold time, the Plasma is clarified. The clarified plasma is adjusted with sodium hydroxide solution to about pH 6.9-7.2.

Preparation of Prothrombin: under agitation and at a temperature of about 0-10° C., about 1.5-2.5 (dry weight) grams of ion exchange resin are added per liter of Bovine Plasma and mixed 0.5-6 hours while controlling the pH at about 6.9-7.2. The resulting suspension is filtered or centrifuged to harvest the resin. The resin is washed thoroughly with 0.15-0.2 molar phosphate buffered saline at about pH 6.9-7.2 and saved. Prothrombin is eluted from the washed resin using 0.5-1 M phosphate buffered saline at a pH of approximately 6.9-7.2, filtered, and the extracts saved and pooled for further processing. Resins that can be used include, but are not limited to, DEAE-Sephadex A-50, Macro-Prep DEAE Support, Macro-Prep High Q Support, Macro-Prep Q Support, UNOsphere Q ion exchange, Capto Q, DEAE-Sepharose Fast Flow, Q Sepharose™ HP or equivalent. The combined extracts may be coarse filtered if desired prior to ultrafiltration. The spent resin may be treated with acid and stored prior to subsequent regeneration and reuse in a similar prothrombin complex manufacturing process.

The Prothrombin Complex extract is concentrated in a suitable ultrafilter system to about 10-50% of the original volume and then diafiltered to remove unwanted salts. The diafiltration is first conducted by adding approximately two to five liters of chilled purified water per liter of concentrate as permeate is removed, and then by adding approximately two to five liters of chilled dilute sodium chloride per liter of concentrate as permeate is removed. The concentrate is then further optionally concentrated and the ultrafiltration completed. The ultrafiltration system is rinsed with several liters of chilled dilute sodium chloride and this wash is added to the concentrate. The resulting Prothrombin Complex is stored at about −15° C. or colder in sealed containers.

Prothrombin Complex is thawed at about 35° C. or less. Prothrombin Complex is diluted to approximately 1,000-5,000 u/mL by addition of purified water containing sufficient calcium chloride to make the final calcium chloride concentration about 0.005-0.03 M. Thromboplastin suspension is added concurrently to the prothrombin complex with the calcium chloride, under gentle agitation. The pH is adjusted to about 7.3 and mixed for about 15-60 minutes. Following activation at about 15-30° C., the suspension is chilled to about ≦10° C.

Activation and pre-purification of thrombin: the activated Prothrombin Complex is diluted to approximately 500-3,000 u/mL with dilute sodium citrate buffer, pH about 6.6. The material may be refiltered as needed.

The pH of the above described mixture is adjusted to about 6.6 by the addition of dilute hydrochloric acid or dilute sodium hydroxide. The activated Prothrombin Complex is added to a cation exchange resin which has been adjusted to a pH of about 6.6. Resins that can be used include, but are not limited to, Amberlite CG-50, Macro-Prep CM Support, Macro-Prep High S Support, Macro-Prep S Support, UNOsphere S ion exchange, SP Sepharose™ HP, Capto S resin or equivalent.

The column is washed with dilute sodium citrate pH 6.6 and then washed with about 0.1 to 0.25 M sodium chloride to remove low affinity proteins which are discarded. This is followed by application of approximately 0.5-1 molar sodium chloride to elute the purified thrombin. The eluate is collected in fractions which are combined according to an in-process assay. The non-sterile bulk may be stored at about 0-10° C. for up to about 48 hours while in-process. The nonsterile bulk thrombin is formulated to no less than about 1000 u/mL by addition of water for irrigation, 30% glycerol, 10% PEG and approximately 0.15-0.3 M sodium chloride. The pH of the formulated thrombin solution is adjusted to pH of about 6.7±1.0 with dilute hydrochloric acid or sodium hydroxide. The formulated non-sterile bulk thrombin may be stored at about 0-110° C. for up to approximately 48 hours prior to sterile processing. The formulated non-sterile bulk thrombin is sterilized by passage through sterile, bacterial retentive non-fiber releasing filters into a suitable sterile holding tank. The resulting product is a highly concentrated α-thrombin which has a MW of about 40 kDa. Samples have an average specific activity of ≧about 1500 u/mg of protein.

In addition, prion clearance studies were performed using an ion exchange chromatographic purification step. The results indicate that the prion clearance level obtained by the thrombin chromatographic purification step is equal to 3.5 logs. The small-scale column used had an inner diameter of 1.6 cm and was packed to a height of 50.2 cm with resin. So the column bed volume was equal to about 101 mL. The packed column was equilibrated with 100 mL of 1 M NaCl followed by 100 mL of 0.025 M Na-citrate pH 6.61. The flow rate of the chromatography system was set at 3.3 mL/min.

The spike sample consisted of 8 mL of 263K Strain Scrapie Hamster Brain Homogenate. The homogenate was sonicated for 20 minutes, then filtered through a 0.45, 0.2, and 0.1 μm filters. After sample spike, a total of 12 mL was taken for the pre-chromatography tests leaving a pre-column spiked sample of 396 mL (400+8−12 mL). The pre-equilibrated column was loaded with the 396 mL of spiked crude thrombin, washed with 144 mL of 0.025 M Na-citrate buffer until eluent absorbance was below 0.4 AU, and washed with 275 mL of 0.2 M NaCl until eluent absorbance was below 0.2 AU. The column was then stripped with 0.65 M NaCl and 37 mL of purified thrombin was collected from the time the absorbance reached 2 AU until the time it fell back to 2 AU.

The pre- and post chromatography samples collected were stored at −60° C. or below prior to performing the prion Western Blot assay. Results are shown in Table 1.

TABLE 1

| Sample | | Sample | Final Titer | Total | Log |
|---|---|---|---|---|---|
| Record | Sample | volume | (log₁₀ | log₁₀ | Reduction |
| Code | Description | (mL) | (PrP^{RES}/mL)) | (PrP^{RES})* | Value |
| 1 | Spiked Load | 396 | 5.8 | 8.4 | 3.5 |
| 2 | Post column | 37 | 3.3 | 4.9 | |

*Total log₁₀ (PrP^{RES}) = Final Titer (log₁₀ (PrP^{RES}/mL)) + log₁₀ (Sample volume (mL))

### Example 2

### Viral Clearance Using Size Exclusion Filtration

The membranes used in this example are Omega™ 1OOK VR and are “cast from modified polyethersulfone on a highly porous polyolefin backing that imparts strength and rigidity to the finished membrane.” The theoretical molecular weight cut-off point is 100 kDa. Passage of small molecules is possible only under tangential flow filtration conditions. Large molecules and viruses are retained by size exclusion. The log reduction value (LRV) for bovine Parvovirus (BPV), which is a very small (20 nm) non-enveloped virus, has been determined to exceed about 3.5 logs.

The thrombin solution evaluated during this viral clearance study is a pre-purified thrombin. Samples typically have a specific activity of greater than about 1500 u/mg of protein. The protein concentration is estimated at approximately 1.2% and the salt concentration at about 0.65 M NaCl. The major component of this thrombin solution is the Active Pharmaceutical Ingredient α-thrombin, which has a molecular weight of approximately 40 kDa. There are several considerations when choosing a panel of model viruses to be included in a viral clearance study. One is to model relevant viruses that have a clear potential of contaminating the starting materials. Another is to include viruses that have a broad range of physical and chemical characteristics, in the panel of model viruses, so that if the virus clearance study shows good clearance of these viruses, then there is assurance that the manufacturing procedure can effectively clear unexpected viral agents.

It is important to consider bovine parvovirus (BPV) because it is a relevant virus that has a clear potential of contaminating the starting materials, and also is extremely small, non-enveloped, and very resistant to physico-chemical treatments. Xenotropic murine leukemia virus (XMuLV), bovine viral diarrhea virus (BVDV), and pseudorabies virus (PRV) are also included as well as BPV. This panel of viruses provides model viruses for the relevant viruses, and provide a good range of physical and chemical characteristics such that clearance of these viruses would suggest that the manufacturing procedure could clear the unexpected agents. The characteristics of the panel of viruses are indicated in Table 2.

TABLE 2
Characteristic summary of the four viruses chosen

| | | | | | Resistance to |
|---|---|---|---|---|---|
| | | | | | physico-chemical |
| Virus | Genome | Envelope | family | Size (nm) | agents |
| BPV | DNA | No | Parvo | 20-25 | High |
| XMuLV | RNA | Yes | Retro | 80-110 | Low |
| PRV | DNA | Yes | Herpes | 150-200 | Medium |
| BVDV | RNA | Yes | Flavi | 40-70 | Medium |

For each of the four viruses considered, two filtration runs are performed: one at a target feed pressure of 8 psi, and the other at a target feed pressure of 12 psi. Each run is performed using a new Omega™ 100K VR membrane.

All runs are performed in a cold room. Each run consists of spiking the sample with 5%, (v/v) of one of the four viruses, filtering through a 0.45 μm filter to remove any virus aggregates, then filtering through the Pall Omega 100K VR membrane. Virus testing is performed on samples taken post spike, post 0.45 μm filtration, and post Omega 100K VR membrane filtration.

Thrombin filtration consists of filtering about 400 mL of pre-purified thrombin through a 0.1 ft² Omega 100K VR membrane. After 80% (320 mL) of the initial thrombin volume is collected in the permeate, the remaining 80 mL retentate still contains a lot of thrombin in addition to viruses and non-thrombin impurities. Continuous diafiltration of this 80 mL solution is used in order to maximize thrombin transmittance. This is achieved by diafiltering the 80 mL with 6× that volume (480 mL) with a NaCl solution.

The final permeate volume is thus twice the volume of the initial thrombin sample. Concentration of this permeate through a 10K VR membrane cassette is then performed to bring back the volume and concentration to the desired level. The purity of the final product is much enhanced as a result of this filtration. For example, the specific activity is increased by more than 30% and the factor Va content is reduced to undetectable levels in the final product as measured by competitive enzyme-linked immunosorbant assay (cELISA).

Virus removal (and large molecule removal) occurs by size exclusion. Very high log reduction values are observed for the 4-virus panel considered (Bovine Parvo Virus, Bovine Viral Diarrhea Virus, Xenotropic Murine Leukemia Virus, and Bovine Pseudorabies Virus). Stability of the post-Omega thrombin product is not compromised due to the increased product purity.

Table 3 summarizes the parameters and conditions of 8 filtration runs. Since the pre-filtration thrombin sample is equal to 400 mL and the filter surface area is equal to 0.1 ft², the ratio of thrombin volume to filter surface area is 4 L/ft². The volume of the virus spike is equal to 20 mL per run (5% v/v). The feed pressure is maintained at 8±2 psi for the first run and 12±2 psi for the second. The retentate pressure is equal to 0±2 psi for all runs.

TABLE 3
Summary of the parameters and conditions of the 8 filtration runs

| | Virus | | | | | | | |
|---|---|---|---|---|---|---|---|---|
| | PRV | | BVD | | BPV | | XmuLV | |
| | Run# | | | | | | | |
| | 1 | 2 | 1 | 2 | 1 | 2 | 1 | 2 |
| Filter surface area (ft) | 0.1 | 0.1 | 0.1 | 0.1 | 0.1 | 0.1 | 0.1 | 0.1 |
| Volume of initial thrombin (mL) | 400 | 400 | 400 | 400 | 400 | 400 | 400 | 400 |
| Volume of initial spiked thrombin (mL) | 420 | 420 | 420 | 420 | 420 | 420 | 420 | 420 |
| Volume of post filtration thrombin (mL) | 820 | 820 | 820 | 820 | 820 | 820 | 820 | 820 |
| Initial cross flow (mL/min) | 41 | 54 | 43 | 54 | 44 | 56 | 43 | 56 |
| Final cross flow at end of filtration (mL/min) | 38 | 51.5 | 40 | 50 | 41 | 55 | 40 | 53.5 |
| Feed Pressure (psi) | 8-10 | 12-14 | 8-10 | 12-14 | 8-10 | 12-14 | 8-10 | 12-14 |
| Retentate pressure (psi) | 0 | 0 | 0 | 0 | 0 | 0 | 0 | 0 |
| Total filtration time (min) | 257 | 214 | 239 | 223 | 236 | 190 | 251 | 204 |
| Pre and post use filter integrity test | Pass | Pass | Pass | Pass | Pass | Pass | Pass | Pass |

For each run, a spiked thrombin sample of 420 mL is filtered through a 0.1 ft² Omega™ 100K VR membrane. When 340 mL of permeate is collected, the remaining 80 mL of retentate solution is diafiltered with 6 times that volume using a 0.65 M NaCl solution. Therefore, the total permeate volume is equal to 340+(6×80)=820 mL. These filtration conditions yield acceptable thrombin recovery as well as enhanced degree of thrombin purity.

The cross flow at the beginning of the 8 psi runs ranges from 41-44 mL/min. Cross-flow filtration is a method of operation in which retained fluid is circulated over the membrane surface which prevent build-up of filtered material on the membrane. The cross flow at the beginning of the 12 psi runs ranges from 54-56 mL/min. The process time for the 8 psi runs ranges from 236-257 min. The process time for the 12 psi runs ranges from 190-223 min. The clearance results of the four different viruses are summarized in Table 4.

TABLE 4
Summary of viral clearance results

| | Virus | | | | | | | |
|---|---|---|---|---|---|---|---|---|
| | PRV | | BVD | | BPV | | XMuLV | |
| Units | Titer + 95% CI | | Titer + 95% CI | | Titer + 95% CI | | Titer + 95% CI | |
| | (Log₁₀PFU/mL) | | (Log₁₀TCID₅₀/mL) | | (Log₁₀TCID₅₀/mL) | | (Log₁₀TCID₅₀/mL) | |
| Run | 1 | 2 | 1 | 2 | 1 | 2 | 1 | 2 |
| LRV per | ≧4.92 | ≧4.92 | 4.35 | 4.22 | 3.83 | 3.62 | 3.86 | 4.47 |
| run | | | | | | | | |

| | | | | |
|---|---|---|---|---|
| Average | ≧4.92 | ≧4.29 | ≧3.74 | ≧4.26 |
| LRV per | | | | |
| Virus | | | | |

The high clearance values achieved and the similarity of the results obtained between the duplicate runs for all of the viruses indicate that the filtration step is robust. The average log reduction values were above 4 for all of the viruses except BPV, which had a log reduction value of 3.74±0.39. Even though this log reduction value was slightly below 4 logs, it is still very high under the set of conditions used.

In addition, prion clearance studies were performed using the size exclusion filtration step. The results indicate that the prion clearance level obtained by the thrombin filtration purification step is equal to 3.6 logs.

The volume of the pre-spike thrombin sample was equal to 400 mL.

The spike sample consisted of 8 mL of 263K Strain Scrapie Hamster Brain Homogenate. The homogenate was sonicated for 20 minutes, then filtered through a 0.45, 0.2, and 0.1 μm filters.

After sample spike, 12 mL were taken for the pre-Omega filtration tests leaving a pre-filtration spiked sample of 396 mL (400+8-12 mL).

The filter used was Pall's Omega™ 100K VR membrane with a surface area of 0.1 ft². So the ratio of thrombin volume to filter surface area was 4 L/ft².

The Omega™ 100K VR membrane was set up on the filtration system and rinsed with 500 mL of purified water. The pre-use integrity test was performed and passed the acceptance criteria. The membrane was then conditioned with 100 mL of 0.65 M NaCl at a feed pressure of 10 psi. Permeate and cross flow rates, measured in graduated cylinders, were about 5 and 52 mL/min respectively.

Filtration of the initial 396 mL of spiked sample was started. When 315 mL of filtrate (i.e. about 80% of the initial volume) was collected, the remaining sample was diafiltered with a total of 475 mL of 0.65 M NaCl (i.e. about 6 times the retentate volume). The feed pressure was maintained at about 10 psi and the retentate pressure was equal to 0 psi throughout the filtration run. These filtration conditions were previously shown to yield acceptable thrombin recovery as well as high virus clearance.

The final post-filtration volume was equal to 790 mL and the process time was equal to 172 minutes.

Pre- and post-Omega filtration samples collected were stored at −60° C. or below prior to performing the prion Western Blot assay. Results are summarized in Table 5.

TABLE 5

| Sample Record | Sample | Sample | Final Titer | Total log₁₀ | Log Reduction |
|---|---|---|---|---|---|
| Code | Description | Volume (mL) | (log₁₀ (PrP^{RES}/mL)) | (PrP^{RES})* | Value |
| 1 | Spiked Load | 396 | 5.7 | 8.3 | 3.6 |
| 2 | Post Omega | 790 | 1.8 | 4.7 | |

*Total log₁₀ (PrP^{RES}) = Final Titer (log₁₀ (PrP^{RES}/mL)) + log₁₀ (Sample volume (mL))

### Example 3

### Viral Clearance Using Ion Filters

Purified thrombin is first concentrated using a Pall filter with 10K MWCO and 1 ft² surface area. Then, the concentrated samples are diluted with purified water to the desired salt concentration. A total of 15 batches are prepared. The results of all the batches prepared are summarized in Tables 6 and 7.

TABLE 6
Average percent recovery for various runs

| | | % | Log Reduction |
|---|---|---|---|
| Run # | Description | Recovery | for BPV |
| 1, 2, & 3 | 50 mM NaCl, 0.8% Mannitol, | 74 | ≧5.12 ± 0.24 |
| | pH ~5.5 | | |
| 11, 12, & | 50 mM NaCl, 0.8% Mannitol, | 76.9 | NA |
| 13 | pH ~7.0 | | |
| 8, 9, & 10 | 72 mM NaCl, 0.8% Mannitol, | 90.8 | NA |
| | pH ~7.0 | | |
| 14, 15, & | 72 mM NaCl, no Mannitol, | 91.7 | 3.60 ± 0.62 |
| 16 | pH ~7.0 | | |
| 4, 5, & 7 | 108 mM NaCl, 0.8% Mannitol, | 99.8 | 1.25 ± 0.55 |
| | pH ~7.0 | | |

TABLE 7
Summary of results

| | Run# | | | | | | | |
|---|---|---|---|---|---|---|---|---|
| | 1 | 2 | 3 | 4 | 5 | 6 | 7 | 8 |
| NaCl | 50 | 50 | 50 | 108 | 108 | 81 | 108 | 72 |
| concentration | | | | | | | | |
| (mM) | | | | | | | | |
| Initial Potency | 30192 | 21332 | 22111 | 32660 | 32660 | 32660 | 27217 | 24107 |
| (u/mL) | | | | | | | | |
| Initial Volume | 600 | 500 | 500 | 400 | 400 | 400 | 400 | 290 |
| (mL) | | | | | | | | |
| Concentration to | 213 | 175 | 151 | 250 | 350 | 350 | 310 | 150 |
| (mL) | | | | | | | | |
| Dilution with | 1:13 | 1:13 | 1:13 | 1:6 | 1:6 | 1:8 | 1:6 | 1:9 |
| H₂O | | | | | | | | |
| Final volume of | 2720 | 2245 | 1930 | 1500 | 2100 | 2800 | 1860 | 1350 |
| formulated | | | | | | | | |
| sample (mL) | | | | | | | | |
| Final potency of | 4650 | 3753 | 4201 | 8562 | 6594 | 4135 | 5559 | 4719 |
| formulated | | | | | | | | |
| sample (u/mL) | | | | | | | | |
| % Recovery | 70 | 79 | 73 | 98.3 | 106 | 88.6 | 95 | 91.1 |
| Mannitol | 0.8 | 0.8 | 0.8 | 0.8 | 0.8 | 0.8 | 0.8 | 0.8 |
| (% w/v) | | | | | | | | |
| pH | 5.49 | 5.51 | 5.53 | 7.04 | 7.05 | 7.02 | 7.03 | 7.10 |
| | Run# | | | | | | | |
| | 9 | 10 | 11 | 12 | 13 | 14 | 15 | 16 |
| NaCl | 72 | 72 | 50 | 50 | 50 | 72 | 72 | 72 |
| concentration | | | | | | | | |
| (mM) | | | | | | | | |
| Initial Potency | 23950 | 29161 | 22928 | 23836 | 24063 | 26569 | 22474 | 23977 |
| (u/mL) | | | | | | | | |
| Initial Volume | 300 | 300 | 300 | 300 | 300 | 300 | 300 | 300 |
| (mL) | | | | | | | | |
| Concentration to | 155 | 145 | 117 | 105 | 115 | 140 | 123 | 125 |
| (mL) | | | | | | | | |
| Dilution with | 1:9 | 1:9 | 1:13 | 1:13 | 1:13 | 1:9 | 1:9 | 1:9 |
| H₂O | | | | | | | | |
| Final volume of | 1395 | 1305 | 1521 | 1365 | 1495 | 1260 | 1107 | 1125 |
| formulated | | | | | | | | |
| sample (mL) | | | | | | | | |
| Final potency of | 4768 | 5954 | 3288 | 4534 | 3454 | 5806 | 5781 | 5658 |
| formulated | | | | | | | | |
| sample (u/mL) | | | | | | | | |
| % Recovery | 92.6 | 88.8 | 72.7 | 86.5 | 71.5 | 91.8 | 94.9 | 88.5 |
| Mannitol | 0.8 | 0.8 | 0.8 | 0.8 | 0.8 | 0 | 0 | 0 |
| (% w/v) | | | | | | | | |
| pH | 7.05 | 7.05 | 7.05 | 7.07 | 7.02 | 6.98 | 6.95 | 7.03 |

Samples from run 1-3 (50 mM NaCl) are initially used for viral clearance validation of the Mustang Q filter and result in very high log reduction value for bovine parvovirus (BPV). However, since the thrombin recovery is low, averaging only 74%, the viral clearance study is repeated using samples from run 4, 5, and 7 (108 mM NaCl), which yield an average thrombin recovery of 99.8% but less than 2 log reduction values for BPV. Finally, the viral clearance study is repeated using samples from run 14, 15, and 16 (72 mM NaCl and 91.7% recovery) and the log reduction value for BPV was acceptable (3.6±0.62).

In addition prion reduction studies were done using an ion exchange filter. The results indicate that the prion clearance level obtained by the thrombin filtration purification step is equal to greater than 3.9 logs.

The volume of the pre-spike thrombin sample was equal to 91 mL.

The spike sample consisted of 1.6 mL of 263K Strain Scrapie Hamster Brain Homogenate. The homogenate was sonicated for 20 minutes, then filtered through a 0.45, 0.2, and 0.1 μm filters. 12 mL were taken for the pre-Mustang Q filtration tests leaving a pre-filtration spiked sample of 80.6 mL (91+1.6−12 mL).

The filter used was Pall's Mustang Q filter with a surface area of 0.35 mL. So the ratio of thrombin volume to filter surface area was 230 mL of sample per mL of filter.

The filter holder was sanitized without filter coin with 20 mL of 1 N NaOH with a 20 min hold. The Mustang Q filter was placed in the holder, washed with 20 mL of 1 N NaOH followed with 1 M NaCl wash until eluent pH was neutral.

Then the filter was conditioned with 25 mL of 72 mM NaCl at a flow rate of about 3 mL/min before the actual filtration of the 80.6 mL spiked sample. The final post-filtration volume was equal to 77 mL and the process time was equal to 49 minutes.

Pre- and post ion filtration samples collected were stored at −60° C. or below prior to performing the prion Western Blot assay. The results are shown in Table 8.

TABLE 8

| | | | | | Log |
|---|---|---|---|---|---|
| Sample | Sample | Sample | Final Titer | Total log₁₀ | Reduction |
| Record Code | Description | Volume (mL) | (log₁₀ (PrP^{RES}/mL)) | (PrP^{RES}) * | Value |
| 1 | Spiked Load | 80.6 | 4.8 | 6.7 | >3.9 |
| 2 | Output | 77 | <0.9 | <2.8 | |

### Example 4

### Size Exclusion Filtration Under Various Conditions

This example also uses size exclusion filtration using Pall Omega 100K VR filters. Three runs are performed at a feed pressure of 8 psi and three are performed at a feed pressure of 12 psi. The parameters of the scaled-down filter are chosen to keep the volume to filter surface area constant, and assure operation in the specified feed pressure range. Each run is performed with a new 0.1 ft² Pall Omega 100K VR filter and all runs are performed in a cold room (≦about 8° C.). A flow meter is included in the system to better monitor the cross-flow during filtration. The flow meter is calibrated in the cold room prior to use.

Table 9 summarizes the conditions and parameters of the six filtration runs. For the 8 psi runs, thrombin activity of the starting material averages 22,091 u/mL and for the final filtrate pool, it averages 11,130 u/mL. The resulting percent of thrombin recovery after 6 diafiltration runs cycles averages 86%. Runs performed at a feed pressure of 8 psi show slightly more thrombin recovery than at 12 psi.

TABLE 9
Summary of thrombin filtration and recovery results

| | Run # 1 | Run # 2 | Run # 3 | Run # 4 | Run # 5 | Run # 6 |
|---|---|---|---|---|---|---|
| Target Feed Pressure | 8 | 8 | 8 | 12 | 12 | 12 |
| Thrombin Volume | 400 | 400 | 400 | 400 | 400 | 400 |
| (mL) | | | | | | |
| Spike Volume (mL) | 20 | 20 | 20 | NA | NA | NA |
| Total volume Pre- | 420 | 420 | 420 | 400 | 400 | 400 |
| filtration sample (mL) | | | | | | |
| Activity Pre-filtration | 22,696 | 24,177 | 19,399 | 21,559 | 21,559 | 20,747 |
| sample (u/mL) | | | | | | |
| Total activity Pre- | 9,532,320 | 10,154,340 | 8,147,580 | 8,623,600 | 8,623,600 | 8,298,800 |
| filtration sample (u) | | | | | | |
| Total volume Post- | 820 | 820 | 820 | 800 | 800 | 800 |
| filtration sample (mL) | | | | | | |
| Activity Post-Filtration | 10,405 | 13,210 | 9,776 | 8,631 | 9,940 | 8,923 |
| sample (u/mL) | | | | | | |
| Total activity Post | 8,532,100 | 10,832,200 | 8,016,320 | 6,904,800 | 7,952,000 | 7,138,400 |
| filtration sample (u) | | | | | | |
| % thrombin recovery | 89.5 | 106.7 | 98.4 | 80.0 | 92 | 86 |
| Total process time | 206 | 219 | 215 | 155 | 173.5 | 166 |

One difference is that at a feed pressure of 12 psi, the cross flow is higher resulting in a faster passage of thrombin, thereby shortening the processing time. Table 10 shows that the filtration step results in a 36.4% increase in thrombin purity or specific activity for the 8 psi runs, and a 37.1% increase for the 12 psi runs. The specific activity increases from a range of 1688.4 to 1986.0 of thrombin/mg protein in the prefiltration samples to a range of 2324.6 and 2690.1 of thrombin/mg protein in the post filtration samples.

TABLE 10
Specific activity of pre- vs. post-Omega 100 filtration samples

| | | Run # 1 | Run # 2 | Run # 3 | Avg | Run # 4 | Run # 5 | Run # 6 | Avg |
|---|---|---|---|---|---|---|---|---|---|
| Target feed pressure | | 8 | 8 | 8 | 8 | 12 | 12 | 12 | 12 |
| (psi) | | | | | | | | | |
| Pre- | Activity | 22,696 | 24,177 | 19,399 | | 21,559 | 21,559 | 20,747 | |
| Omega | (u/mL) | | | | | | | | |
| 100 | Protein | 13.056 | 12.576 | 11.1406 | | 10.8554 | 11.7673 | 12.2881 | |
| Filter | (mg/mL) | | | | | | | | |
| | Specific | 1738.4 | 1922.5 | 1741.3 | 1800.7 | 1986.0 | 1832.1 | 1688.4 | 1835.5 |
| | Activity | | | | | | | | |
| | (u/mg) | | | | | | | | |
| Post | Activity | 10,405 | 13,210 | 9,776 | | 8,631 | 9,940 | 8,923 | |
| Omega | (u/mL) | | | | | | | | |
| 100 | Protein | 4.236 | 5.108 | 4.2055 | | 3.5337 | 3.695 | 3.7365 | |
| Filter | (mg/mL) | | | | | | | | |
| | Specific | 2456.3 | 2586.1 | 2324.6 | 2455.7 | 2,442.5 | 2,690.1 | 2,338.1 | 2490.2 |
| | Activity | | | | | | | | |
| | (u/mg) | | | | | | | | |

| | | | | | | | | |
|---|---|---|---|---|---|---|---|---|
| % increase in | 41.3 | 34.5 | 33.5 | 36.4 | 23.0 | 46.8 | 41.4 | 37.1 |
| Specific Activity | | | | | | | | |
| due to | | | | | | | | |
| nanofiltration | | | | | | | | |

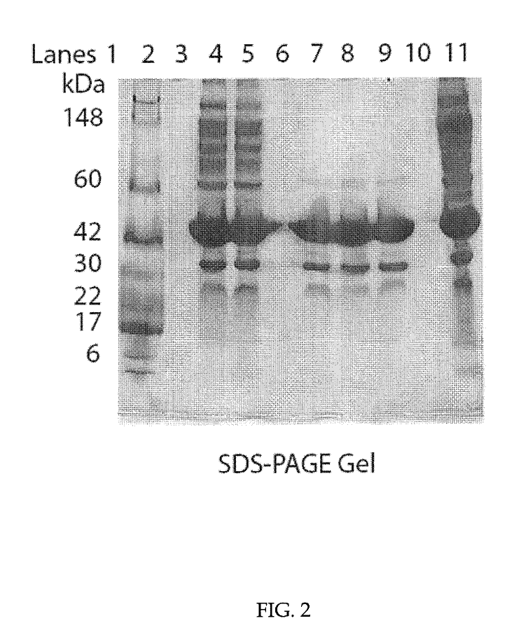

The permeate fractions are much cleaner than the respective initial starting thrombin sample as shown in FIG. 2. Almost all of the high molecular weight impurities observed in the starting material are retained by the filter in the retentate.

Table 11 shows that the filtration step also results in a substantial reduction in Factor Va content. The average reduction between the runs performed at the two feed pressures is comparable: 88.5% in 8 psi runs, and 89.3% in the 12 psi runs. Factor V/Va is associated with coagulopathies that may occur in patients in response to surgical exposure to topical bovine thrombin. Current knowledge suggests that factor V/Va contamination of bovine thrombin stimulates the production of patient antibovine Factor Va antibodies which can cross-react with the patient's own factor Va, thereby leading to impaired hemostasis. This filtration step provides the benefit of substantially reducing factor Va content to undetectable levels in the final thrombin preparation as measured by competitive enzyme-linked immuno sorbant assay (ELISA).

TABLE 11
Factor Va content of pre- vs. post-Omega 100 filtration samples by ELISA

| | | Run # 1 | Run # 2 | Run # 3 | Avg | Run # 4 | Run # 5 | Run # 6 | Avg |
|---|---|---|---|---|---|---|---|---|---|
| Target feed pressure (psi) | | 8 | 8 | 8 | 8 | 12 | 12 | 12 | 12 |
| Pre-Omega | (μg/mL) | 44.993 | 45.566 | 40.879 | 43.813 | 22.954 | 22.954 | 37.163 | 27.690 |
| | (μg/1000 u) | 1.982 | 1.885 | 2.107 | 1.991 | 1.065 | 1.065 | 1.791 | 1.307 |
| Post-Omega | (μg/mL) | 3.625 | 2.017 | 1.801 | 2.481 | 0.741 | 0.708 | 2.357 | 1.269 |
| | (μg/1000 u) | 0.348 | 0.153 | 0.184 | 0.228 | 0.086 | 0.071 | 0.264 | 0.140 |

| | | | | | | | | |
|---|---|---|---|---|---|---|---|---|
| % decrease of Factor Va | 82.4 | 91.9 | 91.3 | 88.5 | 91.9 | 93.3 | 85.3 | 89.3 |
| due to Omega filter | | | | | | | | |

### Example 5

### Stabilized Thrombin Formulation

Table 12 shows a stabilized purified thrombin formulation in accordance with the present invention.

TABLE 12
Stabilized thrombin formulations
Stabilized Thrombin Formulations

| Thrombin Composition | NLT 1,400 units/mL |
|---|---|
| Glycerol | 30% by volume |
| Polyethylene Glycol | 10% by volume |
| Sodium Chloride | 0.15-0.3 M concentration |
| Sodium Acetate | 0.025-0.05 M concentration |
| pH | 6.7 ± 0.1 |

The thrombin for the stabilized thrombin formulation was created according to the process of FIG. 3. The non-sterile bulk thrombin composition was formulated to not less than (NLT) 1,400 units/mL by the addition of water for irrigation. Glycerol was then added to an approximate concentration of 30% by volume. Polyethylene glycol 200-400 MW was added to an approximate 10% concentration be volume. Sodium chloride was added until the concentration of the sodium chloride is approximately 0.15-0.3 M and sodium acetate was added until the concentration of the sodium acetate is approximately 0.025-0.05 M. The pH of the thrombin solution was adjusted to a pH of 6.7±0.1 with dilute hydrochloric acid or sodium hydroxide

The stabilized, non-sterilized, thrombin formulation was then placed on stability testing at room temperature (23° C.±2° C.) and the samples were tested at various time points. The samples were tested in triplicate. The test method used to assay thrombin potency or activity was based on clot time measurements. The machine used to determine clot times was the ACL 7000 coagulation timer. The samples were diluted to a concentration that was within the standard curve range and placed on the machine's rotor. Human plasma was also placed on the machine's cup and both the plasma and diluted thrombin samples were incubated at 37° C. Then a specified volume of each of the plasma and diluted thrombin were concomitantly pumped and mixed. After mixing, light was passed through the plasma/thrombin mixture and when a clot formed the light was scattered. A detector measured the scattered light and transformed the result into clot time (seconds). The clot time was then converted into u/mL using a double log regression of the standard curve's clot time vs. u/mL. Finally, the u/mL value was multiplied by the dilution factor to obtain the actual potency of the sample.

Tables 13 and 14 show the stability results for the formulation of the current invention over 6 months and 24 months, respectively, when stored at room temperature (23° C.±2° C.).

TABLE 13
Stabilized thrombin formulations

| | Label claim | Volume | Initial potency | 3 mo. | 6 mo. |
|---|---|---|---|---|---|
| Sample | u/vial | mL/vial | u/mL | u/mL | u/mL |
| 1 | 5,000 | 5 | 1,661 | 1,613 | 1,611 |
| 2 | 20,000 | 20 | 1,661 | 1,727 | 1,672 |
| 3 | 5,000 | 5 | 1,486 | 1,451 | 1,327 |
| 4 | 20,000 | 20 | 1,486 | 1,483 | 1,492 |

TABLE 14
Stabilized thrombin formulations

| | | | | | | | | | 24 mo. as |
|---|---|---|---|---|---|---|---|---|---|
| | Label | | Initial | | | | | | % of |
| | claim | Volume | potency | 3 mo. | 6 mo. | 9 mo. | 12 mo. | 24 mo. | initial |
| Sample | u/vial | mL/vial | u/mL | u/mL | u/mL | u/mL | u/mL | u/mL | potency |
| 1 | 5,000 | 5 | 1,397 | 1,506 | 1,425 | 1,306 | 1,267 | 1,078 | 77.2 |
| 2 | 10,000 | 10 | 1,484 | 1,592 | 1,357 | 1,346 | 1,288 | 1,068 | 72.0 |
| 3 | 20,000 | 20 | 1,355 | 1,510 | 1,379 | 1,309 | 1,280 | 1,178 | 86.9 |
| 4 | 5,000 | 5 | 1,900 | NA | 1,717 | 1,644 | 1,768 | 1,502 | 79.1 |
| 5 | 5,000 | 5 | 1,765 | NA | 1,433 | 1,471 | 1,564 | 1,258 | 71.3 |
| 6 | 5,000 | 5 | 1,664 | 1,711 | 1,610 | 1,544 | 1,613 | 1,289 | 77.5 |
| 7 | 5,000 | 5 | 1,860 | 1,939 | 1,700 | 1,734 | 1,522 | 1,406 | 75.6 |
| 8 | 5,000 | 5 | 1,451 | 1,480 | 1,347 | 1,348 | 1,229 | 1,106 | 76.2 |

NA = not available

While specific examples have been given, these are preferred embodiments only, and are meant to further explain and describe the invention. They are not intended to define the full scope of this invention.

All references cited herein are incorporated herein by reference in their entirety and for all purposes to the same extent as if each individual publication, patent or patent application was specifically and individually indicated to be incorporated by reference in its entirety for all purposes.

What is claimed is:

1. A process for the preparation of a purified thrombin composition, substantially free of impurities having a molecular weight greater than 40 kDa, and having a specific activity greater than 1800 units of thrombin per mg (u/mg) of protein, which process comprises:
(a) applying a thrombin preparation having from about 500 to about 3000 units of thrombin per mL (u/mL) of the preparation to a cation exchange chromatography to afford a pre-purified thrombin composition having a specific activity greater than about 1500 u/mg of protein;
(b) applying the pre-purified thrombin composition from step (a) to a size exclusion filtration capable of excluding impurities having a molecular weight greater than 40 kDa to afford a purified thrombin composition, substantially free of impurities having a molecular weight greater than 40 kDa, and having a specific activity greater than 1800 u/mg of protein; and optionally,
(c) applying the purified thrombin composition from step (b) to an anion exchange filtration.

2. The process of claim 1, wherein the size exclusion filtration is capable of excluding impurities having a molecular weight greater than 50 kDa.

3. The process of claim 1, wherein the size exclusion filtration is capable of excluding impurities having a molecular weight greater than 100 kDa.

4. The process of claim 1, wherein the size exclusion filtration is capable of excluding impurities having a molecular weight ranging from 40 kDa to 300 kDa.

5. The process of claim 1, wherein the purified thrombin composition has a specific activity between about 1800 and about 3000 u/mg of protein.

6. The process of claim 1, wherein the purified thrombin composition has a specific activity between about 2300 and about 2700 u/mg of protein.

7. The process of claim 1, wherein the purified thrombin composition is substantially free of factor Va.

8. The process of claim 7, wherein the factor Va is present at less than 0.4 μg per 1000 units of thrombin.

9. The process of claim 1, wherein the purified thrombin composition is substantially free of prions.

10. The process of claim 1, wherein the purified thrombin composition is substantially free of viral agents.

11. The process of claim 10, wherein the viral agent is selected from the group consisting of bovine viral diarrhea virus (BVDV), pseudorabies virus (PRV), encephalomyocarditis virus (EMCV), bovine parvovirus (BPV), canine parvovirus (CPV), stickleback virus (SBV), tick-borne encephalitis virus (TBEV), equine rhinovirus 1 (ERV-1), human immunodeficiency virus 1 (HIV-1), hepatitis A virus (HAV), hepatitis B virus (HBV), hepatitis C virus (HCV), and xenotropic murine leukemia virus (XMuLV).

12. The process of claim 1, wherein the purified thrombin composition has a viral clearance log reduction value of at least about 3.5 logs.

13. A purified thrombin composition, substantially free of impurities having a molecular weight greater than 40 kDa, obtainable by the process comprising:
(a) applying a thrombin preparation to a cation exchange chromatography to afford a pre-purified thrombin composition;
(b) applying the pre-purified thrombin composition from step (a) to a size exclusion filtration capable of excluding impurities having a molecular weight greater than 40 kDa to afford a purified thrombin composition, substantially free of impurities having a molecular weight greater than 40 kDa; and optionally,
(c) applying the purified thrombin composition from step (b) to an anion exchange filtration.

14. A purified thrombin composition, substantially free of impurities having a molecular weight greater than 40 kDa, and having a specific activity greater than 1800 units of thrombin per mg (u/mg) of protein obtainable by the process comprising:
(a) applying a thrombin preparation having from about 500 to about 3000 units of thrombin per mL (u/mL) of the preparation to a cation exchange chromatography to afford a pre-purified thrombin composition having a specific activity greater than about 1500 u/mg of protein;
(b) applying the pre-purified thrombin composition from step (a) to a size exclusion filtration capable of excluding impurities having a molecular weight greater than 40 kDa to afford a purified thrombin composition, substantially free of impurities having a molecular weight greater than 40 kDa, and having a specific activity greater than 1800 u/mg of protein; and optionally,
(c) applying the purified thrombin composition from step (b) to an anion exchange filtration.

15. The purified thrombin composition of claim 14, wherein the size exclusion filtration is capable of excluding impurities having a molecular weight greater than 50 kDa.

16. The purified thrombin composition of claim 14, wherein the size exclusion filtration is capable of excluding impurities having a molecular weight greater than 100 kDa.

17. The purified thrombin composition of claim 14, wherein the size exclusion filtration is capable of excluding impurities having a molecular weight ranging from 40 kDa to 300 kDa.

18. The purified thrombin composition of claim 14, wherein the purified thrombin composition has a specific activity between about 1800 and about 3000 u/mg of protein.

19. The purified thrombin composition of claim 14, wherein the purified thrombin composition has a specific activity between about 2300 and about 2700 u/mg of protein.

20. The purified thrombin composition of claim 14, wherein the purified thrombin composition is substantially free of factor Va.

21. The purified thrombin composition of claim 20, wherein the factor Va is present at less than 0.4 μg per 1000 units of thrombin.

22. The purified thrombin composition of claim 14, wherein the purified thrombin composition is substantially free of prions.

23. The purified thrombin composition of claim 14, wherein the purified thrombin composition is substantially free of viral agents.

24. The purified thrombin composition of claim 23, wherein the viral agent is selected from the group consisting of bovine viral diarrhea virus (BVDV), pseudorabies virus (PRV), encephalomyocarditis virus (EMCV), bovine parvovirus (BPV), canine parvovirus (CPV), stickleback virus (SBV), tick-borne encephalitis virus (TBEV), equine rhinovirus 1 (ERV-1), human immunodeficiency virus 1 (HIV-1), hepatitis A virus (HAV), hepatitis B virus (HBV), hepatitis C virus (HCV), and xenotropic murine leukemia virus (XMuLV).

25. The purified thrombin composition of claim 14, wherein the purified thrombin composition has a viral clearance log reduction value of at least about 3.5 logs.
